(19) United States
(12) Patent Application Publication
(10) Pub. No.: US 2017/0107193 A1
(43) Pub. Date: Apr. 20, 2017

(54) CRYSTALLINE LENALIDOMIDE PROCESS

(71) Applicant: SHILPA MEDICARE LIMITED, Karnataka (IN)

(72) Inventors: SRIRAM RAMPALLI, VIZIANAGARAM (IN); LAVKUMAR UPALLA, VIZIANAGARAM (IN); PRASHANT PUROHIT, RAICHUR (IN); AKSHAYKANT CHATURVEDI, RAICHUR (IN)

(73) Assignee: SHILPA MEDICARE LIMITED, KARNATAKA (IN)

(21) Appl. No.: 15/128,994

(22) PCT Filed: Aug. 1, 2014
(86) PCT No.: PCT/IB2014/063614

§ 371 Date: Sep. 26, 2016

(30) Foreign Application Priority Data
Apr. 26, 2014 (IN) 2114/CHE/2014

(51) Int. Cl. C07D 401/04

(52) U.S. Cl. CPC C07D 401/04; C07B 2200/07

(57) ABSTRACT

The present invention relates to process for the preparation of highly pure Lenalidomide (I).

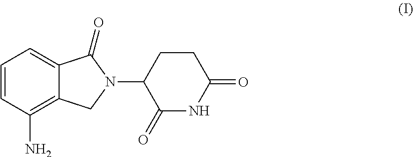 [SMILES: Nc1cccc2c1CN(C1CCC(=O)NC1=O)C2=O]

The invention also relates to crystalline Form-SL obtained by the process of the present invention., the said Form-SL being substantially pure and characterized by X-ray powder diffraction pattern comprising of at least seven peaks selected from 7.061, 12.860, 16.531, 18.698, 27.925, 33.212, 34.187, 35.253, 35.921 and 38.765±0.1°2θ; a single un-split °2θ peak at 7.813±-.1°2θ; and a three-way split °2θ peak at 20.467±0.1°2θ.

The invention further relates to pharmaceutical compositions comprising crystalline Form-SL of Lenalidomide, which may be useful for the treatment of cancer.

### FIELD OF THE INVENTION

The present invention relates to process for the preparation of highly pure Lenalidomide (I).

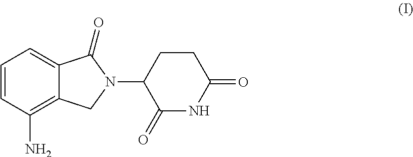 [SMILES: Nc1cccc2c1CN(C1CCC(=O)NC1=O)C2=O]

The present invention also relates to crystalline Form-SL obtained by the process of the present invention, the said Form-SL being substantially pure and characterized by X-ray powder diffraction pattern comprising of at least seven peaks selected from 7.061, 12.860, 16.531, 18.698, 27.925, 33.212, 34.187, 35.253, 35.921 and 38.765±0.1°2θ; a single un-split °2θ peak at 7.813±0.1°2θ; and a three-way-split °2θ peak at 20.467±0.1°2θ.

The present invention further relates to pharmaceutical compositions comprising crystalline Form-SL of Lenalidomide, which may be useful for the treatment of cancer.

### BACKGROUND OF THE INVENTION

Lenalidomide is an immunomodulatory agent with antiangiogenic and antineoplastic properties. Lenalidomide is chemically known as 3-(4-amino4-oxo-1,3-dihydro-2H-isoindol-2-yl)piperidine-2,6-dione of Formula 1.

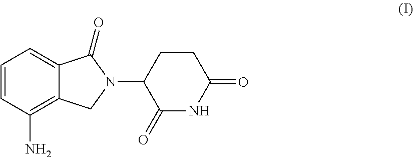 [SMILES: Nc1cccc2c1CN(C1CCC(=O)NC1=O)C2=O]

Lenalidomide has been used to successfully treat both inflammatory disorders and cancers for the past 8 years. Lenalidomide is a thalidomide analogue used in the treatment of multiple cancers and is sold under the trade name REVLIMID® by Celgene.

REVLIMID is indicated for the treatment of patients with i) Multiple myeloma (MM), in combination with dexamethasone, in patients who have received at least one prior therapy; ii) Transfusion-dependent anemia due to low- or intermediate-1-risk myelodysplastic syndromes (MDS) associated with a deletion 5q abnormality with or without additional cytogenetic abnormalities; iii) Mantle cell lymphoma (MCL) whose disease has relapsed or progressed after two prior therapies, one of which included Bortezomib. Lenalidomide is off-white to pale-yellow solid powder known to exist as a hemihydrate form.

Lenalidomide and its process were disclosed initially in U.S. Pat. No. 5,635,517. Further various polymorphic forms of Lenalidomide have been disclosed in US 2005/0096351 A1 by Jaworsky et al. Polymorphic forms of Lenalidomide disclosed in this application are designated as Form-A, Form-B, Form-C, Form-D, Form -E, Form-F, Form-G and Form-H. It is reported in this patent application that the polymorphic Form-A is anhydrous form and polymorphic Form-B is hemi hydrate. Polymorphic Form-C is disclosed to be a hemi-solvate of acetone and Form-D is solvated with water and acetonitrile. Form-E is apparently dihydrate and Form-F is an unsolvated material obtained by dehydration of Form-E. Form-G is also an unsolvated material obtained by slurring Form-B and Form -E in THF solvent. Form-H is a crystalline solid hydrated with about 0.26 moles of water. Lenalidomide Form-B has been described to be the preferred polymorphic form which has been used in the formulation of API into drug product.

Amorphous polymorphic form of Lenalidomide and its methane sulfonic acid salt have been reported in patent application WO 2009/114601 A2. Further strong acid addition salts of Lenalidomide and their polymorphic forms are disclosed in patent application WO 2009/111948 A1, Konakanchi et al in WO2011/111053 Al have disclosed anhydrous Lenalidomide Form I.

Lenalidomide being an important anticancer therapeutic agent, additional and improved ways of preparing Lenalidomide and its polymorphs may be of immense value to pharmaceutical science and the healthcare of cancer patients. Further exploring new forms of pharmaceutically active/useful compounds such as Lenalidomide may provide an opportunity to improve the drug performance characteristics of such products. Hence, there exists a need for the further development of new stable crystalline form of Lenalidomide and economically viable processes for its preparation, which may be commercially up scalable, safer for handling, less time consuming and with better and consistent quality parameters.

The inventors of this application have developed a commercially viable process which provides a stable polymorphic crystalline form of Lenalidomide, designated as Form-SL, which is highly pure, non-hygroscopic and has easy handling properties. The process of this invention provides the crystalline Form-SL of Lenalidomide in a substantially pure form, which is without an detectable impurities/contamination of any other previously known crystalline forms of Lenalidomide.

### OBJECTIVE OF THE INVENTION

The main objective of the invention relates to a process for the preparation of highly pure Lenalidomide.

Another objective of the invention relates to a process for the preparation of highly pure Lenalidomide (I) having purity at least 99.8%.

Yet another objective of the invention relates to crystalline material Form-SL of Lenalidomide characterized by X-ray powder diffraction pattern comprising of at least seven peaks selected from at 7.061, 12.860, 16.531, 18.698, 27.925, 33.212, 34.187, 35.253, 35.921 and 38.765±0.1°2θ; a single un-split °2θ peak at 7.813±0.1°2θ; a three-way-split ′2θ peak at 20.467±0.1°2θ.

### SUMMARY OF THE INVENTION

The main aspect of the invention relates to a process for the preparation of highly pure Lenalidomide (I) (Form-SL), comprising the steps of:

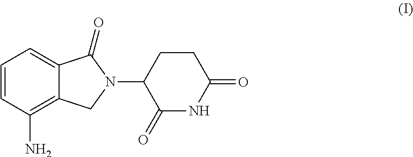 [SMILES: Nc1cccc2c1CN(C1CCC(=O)NC1=O)C2=O]

a) selective hydrogenating compound of formula II

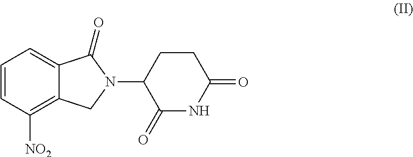 [SMILES: O=C1CCC(N2Cc3c(cccc3[N+](=O)[O-])C2=O)C(=O)N1]

at a hydrogen pressure ranging between 45-60 PSI in methanol;
b) optionally, treating the step (a) material obtained after hydrogenation with a carboxylic acid;
c) concentrate the step (b) solution v/v to 20-50 times of the initial volume; and
d) isolating the pure anhydrous crystalline material (Form-SL).

Another aspect of the invention relates to a process for the preparation of highly pure Lenalidomide (I) having purity at least 99.8% and devoid of impurities A, B and C

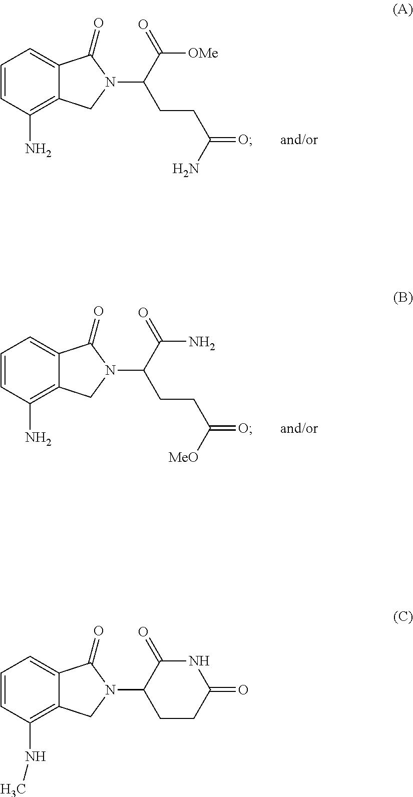 [SMILES: COC(=O)C(CCC(N)=O)N1Cc2c(N)cccc2C1=O ; COC(=O)CCC(C(N)=O)N1Cc2c(N)cccc2C1=O ; CNc1cccc2c1CN(C1CCC(=O)NC1=O)C2=O]

comprising the steps of:
a) adding an organic solvent to Lenalidomide
b) concentrate the reaction mixture by heating up to reflux temperature under atmospheric pressure, in order to active a final volume up to 20-50 times of the initial volume taken, and
c) optionally repeating the steps a) and b).
d) isolating the highly pure Lenalidomide.

In another aspect, the present invention provides crystalline Form-SL Lenalidomide, which is characterized by
i. X-ray powder diffraction pattern comprising of at least seven peaks selected from 7.061, 12.860, 16.531, 18.698, 27.925, 33.212, 34.187, 35.253, 35.921 and 38.765±0.1°2θ; a single un-split °2θ peak at 7.813±0.1°2θ; and a three-way-split °2θ peak at 20.467±0.1°2θ;
ii. DSC isotherm having an endothermic peak at 268° C.;
iii. HPLC purity of at least 99.8%;
iv. Moisture content less than 0.3% (by KF method).

The crystalline Form-SL Lenalidomide as obtained by the process of the present invention is without any detectable impurities/contamination of any other previously known crystalline forms of Lenalidomide.

In a further aspect, the present application also relates to a pharmaceutical composition comprising crystalline Form-SL of Lenalidomide of the present application and at least one or more pharmaceutically acceptable excipients. Such composition is substantially free of any other previously known crystalline forms of Lenalidomide. Further particular aspects of the invention are detailed in the description part of the specification, wherever appropriate.

### BRIEF DESCRIPTION OF THE DRAWINGS

FIG. 1 is an example of X-ray powder diffraction (“XRPD”) pattern of crystalline Form-SL of Lenalidomide

FIG. 2 is an example of a Differential Scanning Calorimetry (“DSC”) curve of Form-SL of Lenalidomide

### DETAILED DESCRIPTION OF THE INVENTION

As set forth herein, embodiments of the present invention relate to a reproducible and efficient process for preparation of crystalline Form-SL Lenalidomide (I) in high yield. Crystalline Form-SL of Lenalidomide obtained by the process of the present invention is found to be substantially pure and stable.

The present invention relates to a process for the preparation of highly pure Lenalidomide (I) (Form-SL), comprising the steps of:

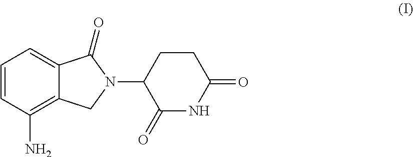 [SMILES: Nc1cccc2c1CN(C1CCC(=O)NC1=O)C2=O]

a) selective hydrogenating compound of formula II

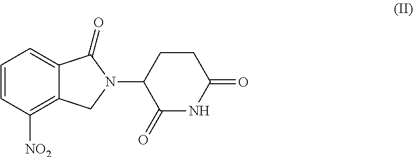 [SMILES: O=C1CCC(N2Cc3c(cccc3[N+](=O)[O-])C2=O)C(=O)N1]

at a hydrogen pressure ranging between 45-60 PSI in methanol;
b) optionally, treating the step (a) material obtained after hydrogenation with a carboxylic acid;
c) concentrate the step (b) solution v/v to 20-50 times of the initial volume; and
d) isolating the pure anhydrous crystalline material (Form-SL).

The individual steps of the process according to the present invention for preparation of highly pure Lenalidomide (I) (Form-SL) are detailed separately herein below.

### Step a) Selective Hydrogenating Compound of Formula II at a Hydrogen Pressure Ranging Between 45-60 PSI in Methanol;

3-(4-nitro-1-oxoisoindolin-2-yl) piperidine-2,6-dione is initially added to 25-75 times (v/w) of methanol to provide a heterogeneous reaction mass. This heterogeneous mass after stirring for 10-20 mins is then added to 150-250 volumes of methanol w.r.t., weight of 3-(4-nitro-1-oxoisoindolin-2-yl)piperidine-2,6-dione initially taken. Optionally the reaction mixture may be degassed by using Nitrogen gas purging for suitable duration of time. The reaction mixture obtained is subjected to hydrogenation in presence of hydrogenation catalyst selected from iron, tin, zinc, palladium, platinum, palladium-carbon, platinum oxide, Raney nickel, samarium, rhodium and ruthenium; at room temperature. Addition of the catalyst to the reaction mixture may be carried out along with continuous nitrogen purging. Treatment of reaction mixture with hydrogen gas may be carried out at 45-60 PSI, which shall be maintained for time duration of 30 mins-4 hrs. Hydrogen gas treatment may be repeated as per the progress of the reaction which may be monitored intermittently by HPLC. Crude Lenalidomide is achieved in reaction mixture at the end of this reaction.

Step b) Optionally, Treating the Step (a) Material Obtained After Hydrogenation with a Carboxylic Acid;

The reaction product obtained from step a) may be subjected to filtration, which may be carried out using any conventional technique known to the person skilled in art, such as but not limiting to the use of celite bed (wherein washing of the celite bed with methanol may also be carried out post filtration). Filtrate provides the solution of the reaction product of step a) with methanol, which is then treated with a carboxylic acid selected from mono carboxylic acid such as formic acid, C2-C5 carboxylic acids selected from acetic acid, propanoic acid, butyric acid, valeric acid; di-carboxylic acid such as oxalic acid, malonic acid, succinic acid, glutaric acid, adipic acid., pimelic acid, maleic acid, fumaric acid, glutaconic acid, traumatic acid, muconic acid, pthalic acid, isopthalic acid, terepthalic acid; tri-carboxylic acid such as citric acid, isocitric acid, aconitic acid, propane-1,2,3-tricarboxylic acid (tricarballylic acid, carballylic acid).

The present inventors found that the formed product was sensitive and observed degradation during the workup of the reaction. By adding catalytic amount of carboxylic acid helps in avoiding the formation of degradation impurities and were controlled by minimizing the formation of impurity.

In view of this, the present inventors overcome the prior-art problems and developed an improved process for the preparation of Lenalidomide using industrially feasible and viable process, with the use of industrially friendly solvents and reagents, which does not include tedious work up.

### Step c) Concentrate the Step (b) Solution v/v to 20-50 Times of the Initial Volume; and

The reaction mixture was concentrated to recover the solvent in order to achieve a final volume up to 20-50 times of the initial volume taken. For clarity, it may be understood that if initial volume of solvent/s is taken up to 3000 ml, the recovery of the solvent shall be such that the volume of recovered solvent shall be at least 2850 ml (i.e. 20 times of the remainder volume of 150 ml in reaction flask). However said recovery volume shall also not increase more than 2940 ml (50 times).

In one of the particular embodiment, such recovery was carried out up to 32 times of the initial volume—which was taken about 3200 ml solvent as initial volume and recovery was carried up to about 3100 ml.

In further embodiment of the present application, another solvent is added to the concentrated reaction mixture obtained after partial recovery of the methanol solvent, thereby providing reaction mass with solvent mixture containing methanol as one of the solvent. The other solvent besides methanol may comprise of organic solvent selected from C₇-C₄ alcohol or C₂-C₅ ester solvent. Among these organic solvents, inventors found that isopropyl alcohol (IPA) or ethyl acetate was found to perform reaction in the similar satisfactory results.

Solvent recovery mentioned above may be optionally carried out under reduced pressure conditions. As per the end product characteristic requirements and matching the desired purity level of the end product, this cycle of addition of solvent to the concentrated reaction mixture and again recovering the solvent to provide concentrated reaction mass may be repeated.

### Step d) Isolating the Pure Anhydrous Crystalline Material (Form-SL).

The concentrated reaction mixture obtained from step c) may be optionally cooled to a temperature of 40° C. or below, if in the previous step its temperature was raised to a higher scale.

The isolation of pure anhydrous crystalline material further comprises of:
a) adding an organic solvent to the wet material obtained in step (c)
b) concentrate the reaction mixture by heating up to reflux temperature under atmospheric pressure, in order to active a final volume up to 20-50 times of the initial volume taken, and
c) optionally repeating the steps a) and b),
d) isolating the pure crystalline material.

The concentrated reaction mixture is filtered under vacuum and given washing with a C₁-C₄ alcoholic such as methanol, ethanol, isopropanol, n-butanol; C₂-C₅ ester solvent or mixtures thereof. The wet solid material is then dried at a temperature of 50-65° C., wherein drying may be optionally carried out under reduced pressure conditions. Drying may be carried out for time duration ranging from 3-20 hrs depending upon achieving the anhydrous end-product as crystalline Form-SL Lenalidomide.

Drying may be also be performed by any conventional process. Drying may be performed under reduced pressure conditions also. Reduced pressure conditions may be suitably utilized by person skilled in the art in order to obtain the dried material. The drying may be performed at a temperature ranging from 50-65° C. for time ranging from 3 to 20 hrs depending upon the physical attributes of the end product obtained i.e. achieving the anhydrous end-product as crystalline Form-SL of Lenalidomide.

In another embodiment the present invention relates to a process for the preparation of highly pure Lenalidomide (I) having purity at least 99.8% and devoid of impurities A, B and C

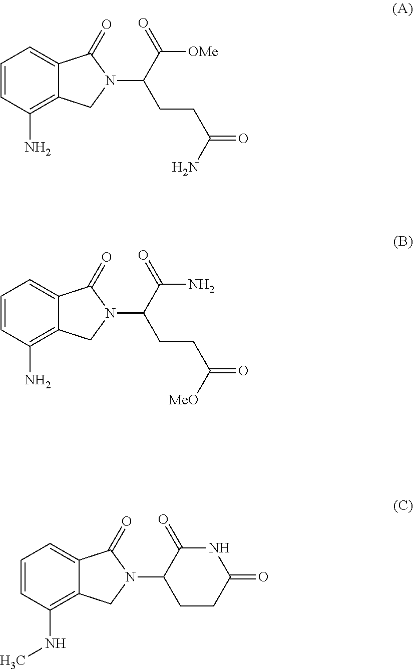 [SMILES: COC(=O)C(CCC(N)=O)N1Cc2c(N)cccc2C1=O ; COC(=O)CCC(C(N)=O)N1Cc2c(N)cccc2C1=O ; CNc1cccc2c1CN(C1CCC(=O)NC1=O)C2=O]

comprising the steps of:
a) adding an organic solvent to Lenalidomide
b) concentrate the reaction mixture by heating up to reflux temperature under atmospheric pressure, in order to active a final volume up to 20-50 times of the initial volume taken, and
c) optionally repeating the steps a) and b).
d) isolating the highly pure Lenalidomide.

The concentrated reaction mixture is filtered under vacuum and given washing with a C₁-C₄ alcoholic such as methanol, ethanol, isopropanol, n-butanol; C₂-C₅ ester solvent or mixtures thereof. The wet solid material is then dried by any conventional process. Drying may be performed under reduced pressure conditions also. Reduced pressure conditions may be suitably utilized by person skilled in the art in order to obtain the dried material. The drying may be performed at a temperature ranging from 50-65° C. for a time ranging from 12 to 16 hours depending upon the physical attributes of the end product obtained i.e. Pure Lenalidomide. The obtained Lenalidomide according to the present invention is having purity greater than 99.8%.

In an another embodiment of the invention, the present invention provides crystalline Form-SL of Lenalidomide, which is characterized by
i. powder diffraction pattern comprising of at least seven peaks selected from 7.061, 12.860, 16.531, 18.698, 27.925, 33.212, 34.187, 35.253, 35.921 and 38.765±0.1°2θ; a single un-split °2θ peak at 7.813±0.1°2θ; and a three-way-split °2θ peak at 20.467±0.1°2θ;
ii. DSC isotherm having an endothermic peak at 268° C.;
iii. HPLC purity of at least 99.8%;
iv. Moisture content less than 0.3% (by KF method).

Crystalline Form-SL Lenalidomide, is a non-hygroscopic crystalline solid, which is further characterized by
i. X-ray powder diffraction pattern comprising of at least seven peaks selected from 7.061, 12.860, 16.531, 18.698, 27.925, 33.212, 34.187, 35.253, 35.921 and 38.765±0.1°2θ; a single un-split °2θ peak at 7.813±0.1°2θ; and a three-way-split °2θ peak at 20.467±0.1°2θ;
ii. DSC isotherm having an endothermic peak at 268° C.

Process of recovering the crystalline Form-SL of Lenalidomide may further comprise processes but not limited to conventional processes including scrapping and if required filtering from slurry which may be carried out at room temperature for the suitable durations to retain the crystalline form characteristics of Form-SL.

The process related impurities that appear in the impurity profile of Lenalidomide may be substantially removed by the process of the present invention resulting in the formation of crystalline Form-SL of high purity, without involving any cumbersome processes like acid-base treatment. The merit of the process according to the present invention resides in that—product isolated after drying is directly obtained as crystalline Form-SL of Lenalidomide. There is no involvement of any prior art known form to obtain the crystalline Form-SL Lenalidomide according to the process of the present invention, thereby reducing any chance of polymorphic contamination.

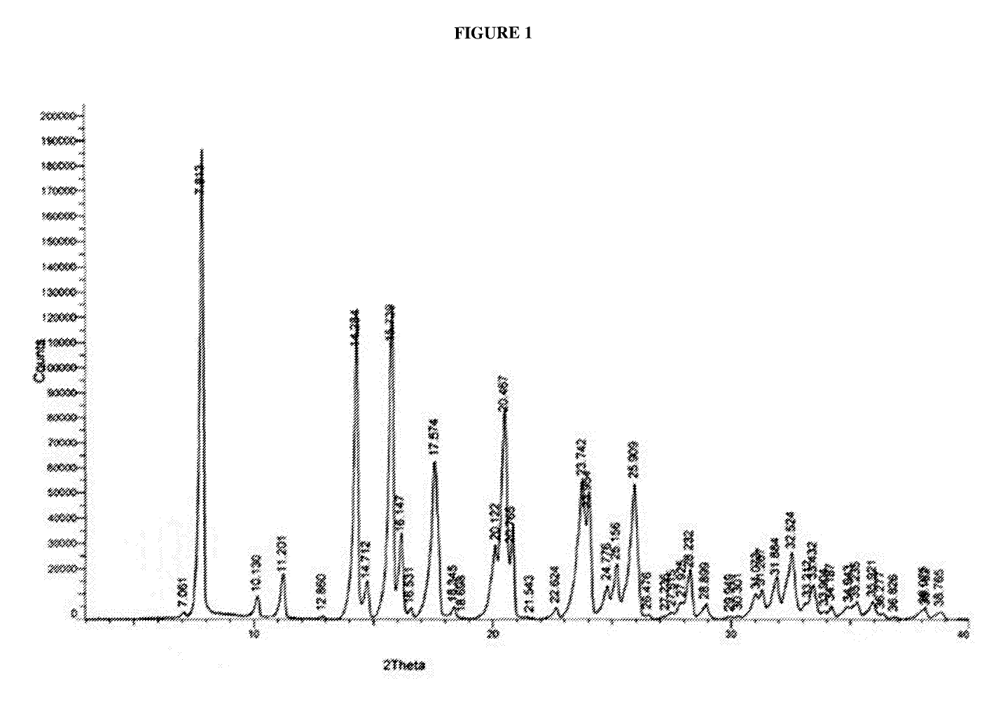

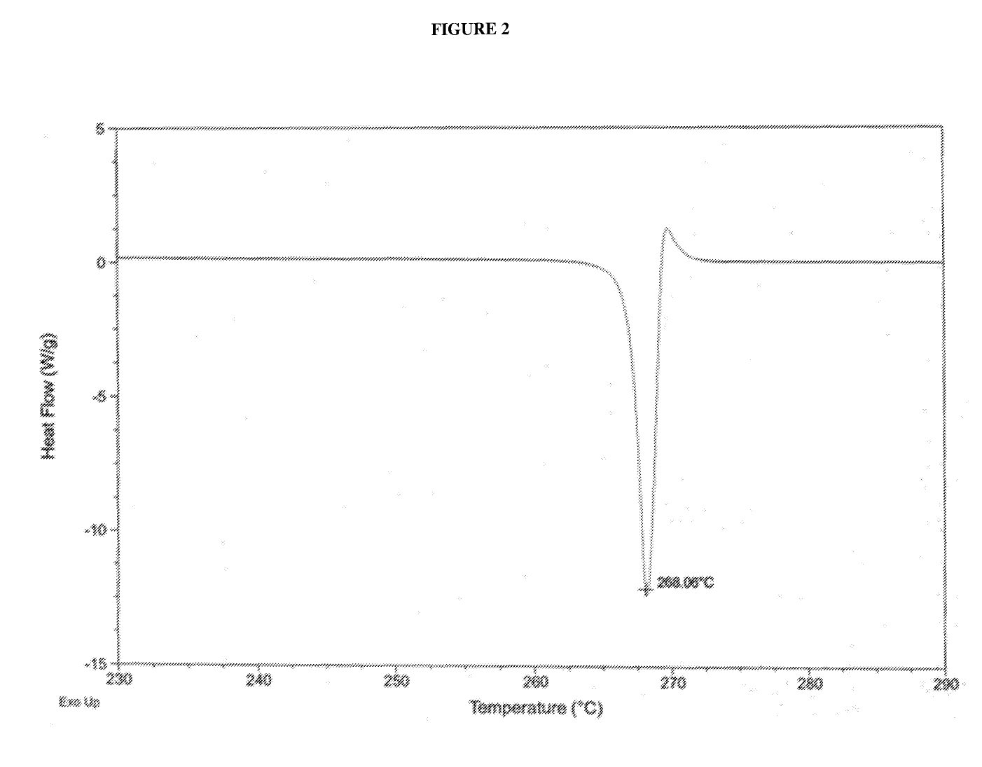

The crystalline Form-SL of Lenalidomide described herein may be characterized by X-ray powder diffraction pattern (XRPD) and Thermal techniques such as differential scanning calorimetry (DSC) analysis. The samples of crystalline Form-SL of Lenalidomide were analyzed by XRPD on a Bruker AXS D8 Advance Diffractometer using X-ray source-Cu Kα radiation using the wavelength 1.5418 Å and lynx Eye detector. DSC was done on a Perkin Elmer Pyris 7.0 instrument. Illustrative examples of analytical data for the crystalline Form-SL of Lenalidomide obtained in the examples are set forth in the FIGS. 1-2.

Another embodiment of the present invention provides crystalline Form-SL of Lenalidomide obtained by the process according to the present invention. Form-SL of Lenalidomide is found adequately stable to handle and store for longer time without any significant or measurable change in its morphology and physicochemical characteristics. Crystalline Form-SL of Lenalidomide obtained according to the process of the present invention is substantially pure i.e. it is found to have final API HPLC purity of more than 99.8% w/w, with moisture content of not more than 0.3% (by KF method).

The crystalline Form-SL of Lenalidomide as obtained by the process of the present invention is without any detectable impurities/contamination of any other previously known crystalline forms of Lenalidomide. This stable form thus, offers various advantages in terms of storage, shelf life, solubility, safety profile, improved physical and/or chemical properties.

In a further embodiment according to this specification, the invention also relates to a composition containing Crystalline Form-SL of Lenalidomide, which is substantially free of any other known forms of Lenalidomide.

In another embodiment the present inventors found that the Lenalidomide obtained as per the present invention is having highly HPLC purity of at least 99.8% and moisture content less than 0.3% (by KF method).

The Crystalline Form-SL of Lenalidomide obtained by the process of the present application may be formulated as solid compositions for oral administration in the form of capsules, tablets, pills, powders or granules. In these compositions, the active product is mixed with one or more pharmaceutically acceptable excipients. The drug substance can be formulated as liquid compositions for oral administration including solutions, suspensions, syrups, elixirs and emulsions, containing solvents or vehicles such as water, sorbitol, glycerin, propylene glycol or liquid paraffin.

In one embodiment of the present invention, it also includes premix comprising one or more pharmaceutically acceptable excipients in the range of 1 to 50% w/w with crystalline Form-SL of Lenalidomide, while retaining the crystalline nature of the premix.

The compositions for parenteral administration can be suspensions, emulsions or aqueous or non-aqueous sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g., using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable excipients used in the compositions comprising crystalline Form-SL of Lenalidomide according to the present application include, but are not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, pre-gelatinized starch and the like; disintegrants such as starch, sodium starch glycolate, pregelatinized starch, Croscarmellose sodium, colloidal silicon dioxide and the like; lubricants such as stearic acid, magnesium stearate, zinc stearate and the like; glidants such as colloidal silicon dioxide and the like; solubility or wetting, enhancers such as anionic or cationic or neutral surfactants, waxes and the like. Other pharmaceutically acceptable excipients that are of use include but not limited to film formers, plasticizers, colorants, flavoring agents, sweeteners, viscosity enhancers, preservatives, antioxidants and the like.

Pharmaceutically acceptable excipients used in the compositions of crystalline. Form-SL of Lenalidomide of the present application may also comprise to include the pharmaceutically acceptable carriers used for the preparation of solid dispersion, wherever utilized in the desired dosage form preparation.

The following examples illustrate the nature of the invention and are provided for illustrative purposes only and should not be construed to limit the scope of the invention.

### EXAMPLES

### Example-01

### Process for Preparation of Lenalidomide

3-(4-nitro-1-oxoisoindolin-2-yl)piperidine-2,6-dione (10 g) and methanol (500 mL) was charged into a 500 mL single necked round bottomed flask and the heterogeneous mass (slurry) was stirred for 10 min. To a clean 5 L autoclave, methanol (2000 mL) and the slurry obtained above were charged with stirring. The reaction mass was degassed for 15 min using Nitrogen purging under stirring. 10% Pd/C (50% wet, 2.0 g) was charged in to the reaction flask with nitrogen purging and then Methanol (500 mL) was charged in to the autoclave. The reaction mixture was then degassed twice with Hydrogen at 50-55 PSI. The reaction mixture was maintained at 50-55 PSI Hydrogen pressure for about 30 min and then the Hydrogen pressure was released. On completion of the reaction as confirmed by intermittent reaction monitoring by HPLC, the reaction mixture was filtered through celite bed and washed with Methanol (200 mL). The filtrate was concentrated at 50° C. under vacuum of 260-360 mm of Hg till about only 200 mL vol. of reaction mixture is left. The reaction mass was then filtered under vacuum, washed with Methanol (50 mL) and suck dried for 1 h. The solid material was dried at 55° C. under vacuum (10-15 mm of Hg) for about 12 h, to obtain Lenalidomide having XRPD similar to FIG. 1 and DSC thermogram similar to FIG. 2.

Yield: 5.9 g

HPLC purity: 99.80%

### Example-02

### Process for Preparation of Lenalidomide

3-(4-nitro-1-oxoisoindolin-2-yl)piperidine-2,6-dione, (10 g) and methanol (500 mL) was charged into a single necked round bottomed flask and the heterogeneous mass (slurry) was stirred for 15 min. To a clean 5 L autoclave, methanol. (2000 mL) and the slurry obtained above were charged with stirring. The reaction mass was degassed for 20 min using Nitrogen purging under stirring. 10% Pd/C (50% wet, 2.0 g) was charged in to the reaction flask with nitrogen purging and then Methanol (500 mL) was charged in to the autoclave. The reaction mixture was then degassed thrice with Hydrogen at 50-55 PSI. The reaction mixture was maintained at 50-55 PSI Hydrogen pressure for about 35 min and then the Hydrogen pressure was released. On completion of the reaction as confirmed by intermittent reaction monitoring by HPLC, the reaction mixture was filtered through celite bed and washed with Methanol (200 mL). The filtrate was concentrated at 55° C. under vacuum of 260-360 mm of Hg till about only 200 mL vol. of reaction mixture is left. To this reaction mixture, isopropyl alcohol (200 mL) was added and again concentration of the reaction mixture was carried out under atmospheric pressure at 80° C. till about only 200 mL vol. of reaction mixture is left. The reaction mass was cooled to 40° C., filtered under vacuum with nitrogen blanket, washed with isopropyl alcohol (50 mL) and suck dried for 1 h. The solid material was further dried at 60° C. under vacuum (10-15 mm of Hg) for about 15 h, to obtain Lenalidomide having XRPD similar to FIG. 1 and DSC thermogram similar to FIG. 2.

6.9 g

HPLC purity: ˜99.81%

### Example-03

### Purification of Lenalidomide

Lenalidomide (7 g) obtained according to example 1/example 2, Methanol (2000 mL) citric acid (1.33 g) was charged in to a round bottomed flask and heated the reaction mixture to 60-65° C. till clear solution was obtained. Concentrate the reaction mass under vacuum till about only 100 mL vol. of reaction mixture is left. Filtered the material and washed with methanol (50 ml). The wet material obtained is charged in to a reaction flask containing isopropyl alcohol (120 mL) and again concentrate the reaction mass under atmospheric pressure at 80° C. till about only 50 mL vol. of reaction mixture is left. This procedure was repeated once/twice again. The obtained material was filtered at 50-55° C. under vacuum with nitrogen blanket, washed with isopropyl alcohol (50 mL) and suck dried for 1 h. The solid material was further dried at 50-60° C. under vacuum (10-15 mm of Hg) for about 15 h, to obtain the highly pure crystalline Lenalidomide having XRPD similar to FIG. 1. (designated as Form-SL) and DSC thermogram similar to FIG. 2 (designated as Form-SL).

Yield: 4.88 g

HPLC purity: ˜99.90%

Moisture Content: <0.3% (by KF)

### Example-04

### Process for Preparation of Lenalidomide

3-(4-nitro-1-oxoisoindolin-2-yl)piperidine-2,6-dione (10 g) and methanol (500 mL) was charged into a 1000 mL single necked round bottomed flask and the heterogeneous mass (slurry) was stirred for 15 min. To a clean 5 L autoclave, methanol (2500 mL) and the slurry obtained above were charged with stirring. The reaction mass was degassed for 20 min using Nitrogen purging under stirring. 10% Pd/C (50% wet, 2.0 g) was charged in to the reaction flask with nitrogen purging. The reaction mixture was then degassed thrice with Hydrogen at 50-55 PSI. The reaction mixture was maintained at 50-55 PSI Hydrogen pressure for about 2 h and then the Hydrogen pressure was released. On completion of the reaction as confirmed by intermittent reaction monitoring by HPLC, the reaction mixture was filtered through celite bed and washed with Methanol (200 mL). Citric acid (670 mg) was charged in to the reaction flask. The filtrate was concentrated at below 40° C. under vacuum of 260-360 mm Hg till about only 100 mL vol. of reaction mixture is left. Filtered the mass and washed with 50 ml of Methanol. To the obtained wet solid, isopropyl alcohol (100 mL) was added and again concentration of the reaction mixture was carried out under atmospheric pressure at 80° C. till about only 50 mL vol. of reaction mixture is left. This procedure was repeated once/twice again. The obtained reaction mass was cooled to 40° C., filtered under vacuum with nitrogen blanket, washed with isopropyl alcohol (50 mL) and suck dried for 1 h. The solid material was further dried at 50-60° C. under vacuum (10-15 mm of Hg) for about 15 h, to obtain highly pure crystalline Lenalidomide having XRPD similar to FIG. 1 (designated as Form-SL) and DSC thermogram similar to FIG. 2 (designated as Form-SL).

Yield: 4.6 g

HPLC purity: ˜99.90%

Moisture Content: <0.24% (by KF)

While the foregoing pages provide a detailed description of the preferred embodiments of the invention, it is to be understood that the description and examples are illustrative only of the principles of the invention and not limiting. Furthermore, as many changes can be made to the invention without departing from the scope of the invention, it is intended that all material contained herein be interpreted as illustrative of the invention and not in a limiting sense.

1) A process for the preparation of highly pure Lenalidomide (I) (Form-SL), comprising the steps of: 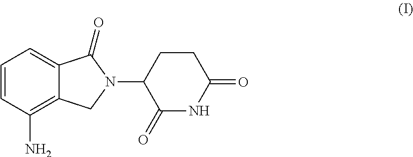 [SMILES: Nc1cccc2c1CN(C1CCC(=O)NC1=O)C2=O]
a) selective hydrogenating compound of formula II 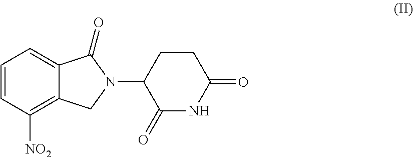 [SMILES: O=C1CCC(N2Cc3c(cccc3[N+](=O)[O-])C2=O)C(=O)N1]
at a hydrogen pressure ranging between 45-60 PSI in methanol;
b) optionally, treating the step (a) material obtained after hydrogenation with a carboxylic acid;
c) concentrating the step (b) solution v/v to 20-50 times of the initial volume; and
d) isolating the pure anhydrous crystalline material (Form-SL).

2) A process for the preparation of highly pure Lenalidomide (I) according to claim 1, wherein selective hydrogenation is carried out in presence of a hydrogenation catalyst selected from iron, tin, zinc, palladium, platinum, palladium-carbon, platinum oxide, Raney nickel, samarium, rhodium and ruthenium.

3) A process for the preparation of highly pure Lenalidomide (1) according to claim 1, wherein carboxylic acid is selected from mono carboxylic acid such as formic acid, C2-C5 carboxylic acids selected from acetic acid, propanoic acid, butyric acid, valeric acid; di-carboxylic acid such as oxalic acid, malonic acid, succinic acid, glutaric acid, adipic acid, pimelic acid, maleic acid, fumaric acid, glutaconic acid, traumatic acid, muconic acid, pthalic acid, isopthalic acid, terepthalic acid; tri-carboxylic acid such as citric acid, isocitric acid, aconitic acid, propane-1,2,3-tricarboxylic acid (tricarballylic acid, carballylic acid).

4) A process for the preparation of highly pure Lenalidomide (I) according to claim 1, wherein step (d) of isolating the pure anhydrous crystalline material further comprises of:
a) adding an organic solvent selected from alcohol such as methanol, ethanol, isopropanol, n-butanol; C₂-C₅ ester solvent or mixtures thereof; to the wet material obtained in step (c);
b) concentrate the reaction mixture by heating up to reflux temperature under atmospheric pressure, in order to active a final volume up to 20-50 times of the initial volume taken;
c) optionally repeating the steps a) and b); and
d) isolating the pure crystalline material.

5) (canceled)

6) A process for the preparation of highly pure Lenalidomide (I) having purity at least 99.8% and devoid of impurities A, B and C 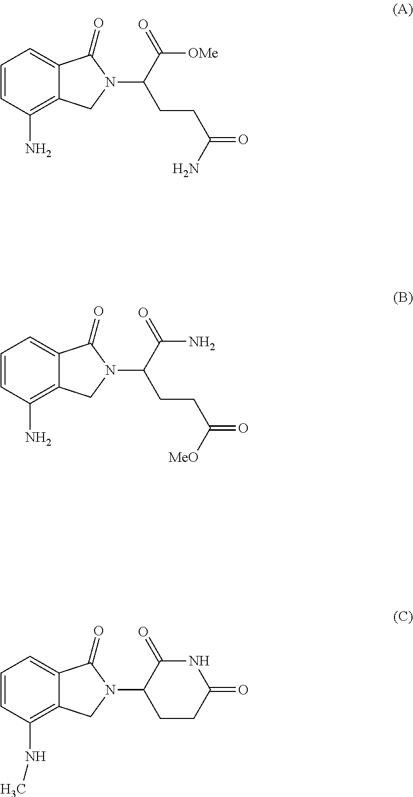 [SMILES: COC(=O)C(CCC(N)=O)N1Cc2c(N)cccc2C1=O ; COC(=O)CCC(C(N)=O)N1Cc2c(N)cccc2C1=O ; CNc1cccc2c1CN(C1CCC(=O)NC1=O)C2=O]
comprising the steps of:
a) adding an organic solvent to Lenalidomide;
b) concentrate the reaction mixture by heating up to reflux temperature under atmospheric pressure, in order to active a final volume up to 20-50 times of the initial volume taken;
c) optionally repeating the steps a) and b); and
d) isolating the highly pure Lenalidomide.

7) Crystalline material Form-SL of Lenalidomide characterized by X-ray powder diffraction pattern comprising of at least seven peaks selected from 7.061, 12.860, 16.531, 18.698, 27.925, 33.212, 34.187, 35.253, 35.921 and 38.765±0.1°2θ; a single un-split °2θ peak at 7.813±0.1°2θ; a three-way-split °2θ peak at 20.467±0.1°2θ.

8) Crystalline material Form-SL of Lenalidomide according to claim 7, further characterized by DSC isotherm having an endothermic peak at 268° C.

9) Highly pure crystalline Form-SL of Lenalidomide according to claim 1, with HPLC purity of at least 99.8% and moisture content less than 0.3% w/w (by KF method).

10) A pharmaceutical composition comprising crystalline Form-SL of Lenalidomide according to claim 6, and at least one or more pharmaceutically acceptable excipients.
